(19) United States
(12) Patent Application Publication
(10) Pub. No.: US 2024/0050427 A1
(43) Pub. Date: Feb. 15, 2024

(54) TREATMENTS FOR OBSESSIVE COMPULSIVE DISORDER

(71) Applicant: SIRGARTAN HOLDINGS LTD, London (GB)

(72) Inventors: Vincenzo GARZYA, London (GB); Jon TANCOCK, London (GB); Nicolas Thierry SIREAU, London (GB); Trevor ROBBINS, London (GB)

(21) Appl. No.: 18/364,827

(22) Filed: Aug. 3, 2023

(30) Foreign Application Priority Data
Aug. 3, 2022 (GB) 2211336.9

(51) Int. Cl. A61K 31/496; A61P 25/18

(52) U.S. Cl. CPC A61K 31/496; A61P 25/18

(57) ABSTRACT

The present invention provides for an effective pharmacological therapy for obsessive compulsive disorder.

### CROSS-REFERENCE TO RELATED APPLICATION

This application claims the benefit of Great Britain Application No. 2211336.9 filed Aug. 3, 2022, the entire contents of which are incorporated herein by reference in its entirety.

The present invention relates to the treatment of obsessive compulsive disorder (OCD).

### BACKGROUND OF THE INVENTION

Obsessive compulsive disorder (OCD) is a chronic and debilitating neuropsychiatric condition where a patient has intrusive thoughts or images (obsessions) and/or repetitive or ritualistic actions (compulsions). These repetitive or ritualistic actions are performed in an attempt to alleviate stress resulting from the intrusive thoughts or images. Whilst most people may have some obsessions and compulsions, those with OCD have no control of their obsessions and compulsions to the point that that they are unable to carry out everyday tasks and their lives are negatively impacted.

The obsessions are not simply excessive worries about real life issues. The thoughts and images are intrusive, inappropriate and cause anxiety or distress (Stein, The Lancet, 360, 3 Aug. 2002). The compulsions result from the patient trying to suppress such thoughts through another thought or action. Such compulsions are aimed at reducing distress or preventing a dreaded situation from occurring but are not connected in a realistic way with what they are intended to neutralise or are clearly excessive. The compulsions are time-consuming and interfere with the patient's social and occupational activities. Typically, those with OCD are aware that their obsessions are unreasonable and this causes further distress. One manifestation of OCD involves patients engaging in excessive checking behaviour, such as checking if doors are locked or appliances are switched off. Checking behaviour itself can be useful, but in OCD the excessive checking shown by patients becomes maladaptive and are performed at the expense of other activities. The compulsive checking may be due to obsessions about safety (such as fears about being responsible for a fire, flood or some sort of fatal accident), health, mistakes, or inappropriate behaviour concerns.

OCD is common. The worldwide prevalence is estimated to be between 1-3% of the adult population and thus OCD represents a major health-economic burden. Despite this high prevalence, the causes of OCD are unknown. There has been some evidence to suggest that genetics play a role, but work in this field has not yielded any definitive molecular targets for therapeutic intervention (Robbins et al., Neuron 102, 3 Apr. 2019). There is also evidence that exogenous factors can cause acute exacerbations in OCD, which may arise for instance either from a stress trigger eliciting a psychological disturbance or from an infection. Accordingly, current therapeutic approaches to treating OCD involve psychological therapy and limited pharmacotherapy. Selective serotonin reuptake inhibitors (SSRIs) are the first line pharmacological treatment of OCD.

SSRIs work by increasing the amount of the neurotransmitter serotonin in the synapse. Serotonin is released by the presynaptic cell and then diffuses across the synapse to the postsynaptic cell, where it binds to serotonin receptors to stimulate neuronal signalling in that cell. Once the serotonin is released and the message transmitted, the serotonin is taken back up by the presynaptic cell to be reused. SSRIs increase the amount of serotonin in the synapse by inhibiting serotonin reuptake by the presynaptic cell. This increases the amount of serotonin in the synapse and thus increases stimulation of the serotonin receptors on the postsynaptic cell.

SSRIs are usually used to treat major depressive disorders, but they exhibit utility in treating OCD when used at high doses, typically above the dose range established by their manufacturers. Consequently, the risk of side effects is increased. For example, the Food and Drug Administration has issued a warning against the use of the SSRI citalopram in doses in excess of 40 mg/day due to an increased risk of heart complications, and high levels of patient dropout due to negative side-effects was observed in a clinical trial investigating SSRIs for the treatment of OCD (Bloch et al., Mol. Psychiatry, 2010, 15(8), 850-855). Furthermore, the efficacy of SSRIs in treating OCD is low, meaning that approximately half of OCD patients do not respond sufficiently to SSRIs (Kellner, Dialogues in Clinical Neuroscience, 12, 2, 2010).

Finally, the clinical effect of SSRIs is normally only achieved 2-3 months after starting medication, which itself normally arises from a medical consultation that results from a mental health crisis. During this period, the OCD patient is at high risk of self-harm, and may attempt suicide, sometimes successfully. Accordingly, there exists a need for more effective pharmacological therapies for OCD with fewer side effects and more rapid action.

### SUMMARY OF THE INVENTION

In a first aspect the invention provides a compound comprising, or having, the structure:

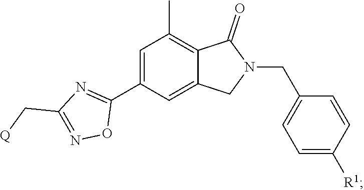

wherein:
   R¹ is halo or C₁₋₃ haloalkoxyl;
Q is

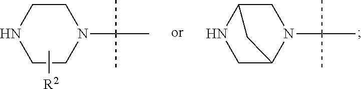

and
R² is hydrogen or C₁₋₃ alkyl,
or a pharmaceutically acceptable salt, hydrate, solvate, optical isomer or polymorph thereof, for use in the treatment of obsessive compulsive disorder (OCD) in a patient. Preferably, the salt is a hydrochloride salt, a mesylate salt or a valine salt. Even more preferably, the salt is a mesylate salt.
R¹ may be chloro or trifluoromethoxyl. Preferably R¹ is trifluoromethoxyl.
Preferably, Q is

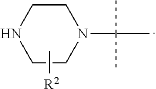

Preferably, R² is hydrogen.

Preferably, R¹ is trifluoromethoxyl, Q is

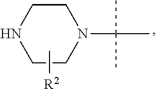

and R² is hydrogen.

Preferably, the compound comprises, or has, the structure:

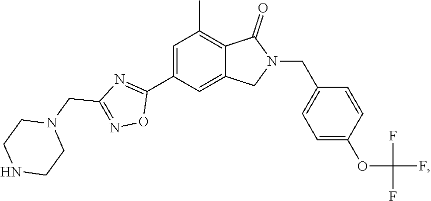 [SMILES: Cc1cc(-c2nc(CN3CCNCC3)no2)cc2c1C(=O)N(Cc1ccc(OC(F)(F)F)cc1)C2]

or a pharmaceutically acceptable salt, hydrate, solvate, optical isomer, or polymorph thereof.

Preferably, the compound comprises, or is, 7-methyl-5-(3-piperazin-1-ylmethyl-[1,2,4]oxadiazol-5-yl)-2-(4-trifluoromethoxybenzyl)-2,3-dihydroisoindol-1-one, also known as AZD-8529, or comprises a pharmaceutically acceptable salt, hydrate, solvate, optical isomer, or polymorph thereof.

The compound or the pharmaceutically acceptable salt, hydrate, solvate, optical isomer, or polymorph thereof may be administered at a dose of 0.01 mg/kg to 100 mg/kg, 0.05 mg/kg to 50 mg/kg, 0.1 mg/kg to 30 mg/kg, 0.5 mg/kg to 10 mg/kg, or preferably 0.5 mg/kg to 5 mg/kg, preferably 2 mg/kg to 4 mg/kg, more preferably 2.5 mg/kg to 3.5 mg/kg. The compound the pharmaceutically acceptable salt, hydrate, solvate, optical isomer, or polymorph thereof may be administered at a dose of at least 1 mg/kg, at least 2 mg/kg, at least 0.5 mg/kg, at least 4 mg/kg, at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 10.5 mg/kg, at least 14 mg/kg, at least 15 mg/kg, at least 16 mg/kg, at least 17 mg/kg, at least 18 mg/kg, at least 19 mg/kg, at least 20 mg/kg, at least 25 mg/kg, at least 30 mg/kg, at least 35 mg/kg, at least 40 mg/kg, at least 45 mg/kg, or at least 50 mg/kg. The compound the pharmaceutically acceptable salt, hydrate, solvate, optical isomer, or polymorph thereof may be administered at a dose of at most 1 mg/kg, at most 2 mg/kg, at most 0.5 mg/kg, at most 4 mg/kg, at most 5 mg/kg, at most 6 mg/kg, at most 7 mg/kg, at most 8 mg/kg, at most 9 mg/kg, at most 10 mg/kg, at most 11 mg/kg at most 12 mg/kg, at most 10.5 mg/kg, at most 14 mg/kg, at most 15 mg/kg, at most 16 mg/kg, at most 17 mg/kg, at most 18 mg/kg, at most 19 mg/kg, at most 20 mg/kg, at most 25 mg/kg, at most 30 mg/kg, at most 35 mg/kg, at most 40 mg/kg, at most 45 mg/kg, or at most 50 mg/kg.

The compound or the pharmaceutically acceptable salt, hydrate, solvate, optical isomer, polymorph thereof may be administered in a dose of 30 mg to 40 mg, preferably 33 mg to 37 mg, such as about 35 mg. Alternatively, the pharmaceutical composition may comprise the compound or the pharmaceutically acceptable salt, hydrate, solvate, optical isomer, polymorph thereof in an amount of 35 mg to 45 mg, preferably 38 mg to 43 mg, such as about 40 mg.

The compound or the pharmaceutically acceptable salt, hydrate, solvate, optical isomer, polymorph thereof may be administered in a dose of 40 mg, 45 mg, 50, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 140 mg, or 150 mg.

The compound or the pharmaceutically acceptable salt, hydrate, solvate, optical isomer, polymorph thereof may be administered in a dose of 40 mg to 50 mg, 45 mg to 55 mg, 50 mg to 60 mg, 55 mg to 65 mg, 60 mg to 70 mg, 65 mg to 75 mg, 70 mg to 80 mg, 75 mg to 85 mg, 80 mg to 90 mg, 85 mg to 95 mg, 90 mg to 100 mg, 95 mg to 105 mg, 100 mg to 110 mg, 105 mg to 115 mg, 110 mg to 120 mg, 115 mg to 125 mg, 120 mg to 130 mg, 125 mg to 135 mg, 130 mg to 140 mg, 135 mg to 145 mg, or 140 mg to 150 mg.

Suitably, the aforementioned dosages are administered once, twice or three time per day, preferably once or twice per day, more preferably once per day. Alternatively, the dosages may be administered once every 2 days (i.e., every second day).

The compound or the pharmaceutically acceptable salt, hydrate, solvate, optical isomer, or polymorph thereof may be administered orally, parenterally, subcutaneously, intravenously, intramuscularly, intrathecally, intradermally, intraarterially, intraarticularly, intraperitoneally, via cutaneous administration, via transcutaneous administration, via intra-osseus administration or by inhalation. Oral administration is preferred.

The patient's OCD may be characterised by a score according to the Yale-Brown Obsessive Compulsive scale of at least 8, at least 16, a least 24 or optionally at least 32. The patient's OCD may be characterised by a Yale-Brown Obsessive Compulsive scale score of 16-40, 24-40 or optionally 32-40.

Alternatively, or additionally, the patient's OCD may be characterised a score according to the Obsessive-Compulsive Inventory (OCI) or Obsessive-Compulsive Inventory—Revised (OCI-R) of at least 21, at least 30, at least 40, at least 50, or at least 60. The patient's OCD may be characterised a score according to the Obsessive-Compulsive Inventory (OCI) or Obsessive-Compulsive Inventory—Revised (OCI-R) of 21-72, 30-72, 40-72, 50-72 or 60-72.

The patient may have checking compulsions.

In a second aspect the invention provides a pharmaceutical composition for use in the treatment of obsessive compulsive disorder in a patient, wherein said pharmaceutical composition comprises the aforementioned compound or a pharmaceutically acceptable salt, hydrate, solvate, optical isomer, or polymorph thereof.

The pharmaceutical composition may comprise the compound or the pharmaceutically acceptable salt, hydrate, solvate, optical isomer, polymorph thereof in an amount of 0.7 mg to 7000 mg, 3.5 mg to 3500 mg, 7 mg to 2100 mg, 21 mg to 700 mg, preferably 20 mg to 210 mg, 25 mg to 190 mg, more preferably 30 mg to 170 mg, 35 mg to 150 mg. Even more preferably, the compound is administered at a dose of 140 mg.

The pharmaceutical composition may comprise the compound or the pharmaceutically acceptable salt, hydrate, solvate, optical isomer, polymorph thereof in an amount of 30 mg to 40 mg, preferably 33 mg to 37 mg, such as about 35 mg. Alternatively, the pharmaceutical composition may comprise the compound or the pharmaceutically acceptable salt, hydrate, solvate, optical isomer, polymorph thereof in an amount of 35 mg to 45 mg, preferably 38 mg to 43 mg, such as about 40 mg.

The pharmaceutical composition may comprise the compound or the pharmaceutically acceptable salt, hydrate, solvate, optical isomer, polymorph thereof in an amount of 40 mg, 45 mg, 50, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 140 mg, or 150 mg.

The pharmaceutical composition may comprise the compound or the pharmaceutically acceptable salt, hydrate, solvate, optical isomer, polymorph thereof in an amount of 40 mg to 50 mg, 45 mg to 55 mg, 50 mg to 60 mg, 55 mg to 65 mg, 60 mg to 70 mg, 65 mg to 75 mg, 70 mg to 80 mg, 75 mg to 85 mg, 80 mg to 90 mg, 85 mg to 95 mg, 90 mg to 100 mg, 95 mg to 105 mg, 100 mg to 110 mg, 105 mg to 115 mg, 110 mg to 120 mg, 115 mg to 125 mg, 120 mg to 130 mg, 125 mg to 135 mg, 130 mg to 140 mg, 135 mg to 145 mg, or 140 mg to 150 mg.

Suitably, the aforementioned pharmaceutical compositions are administered once, twice or three time per day, preferably once or twice per day, more preferably once per day. Alternatively, the pharmaceutical compositions may be administered once every 2 days (i.e., every second day).

The pharmaceutical composition may be administered orally, parenterally, subcutaneously, intravenously, intramuscularly, intrathecally, intradermally, intraarterially, intraarticularly, intraperitoneally, via cutaneous administration, via transcutaneous administration, via intra-osseus administration or by inhalation. Oral administration is preferred.

As demonstrated in the Examples whereby the rats undertook the behavioural tests 2 hours after dosing, the inventors have surprisingly found that the compounds of the invention have a fast onset of action, unlike SSRIs which often take at least 3 months to result in any effects. This is particularly advantageous in the treatment of OCD, whereby often patients are high risk, particularly of self-harm or suicide. Suitably the compound or the pharmaceutically acceptable salt, hydrate, solvate, optical isomer, or polymorph thereof, or the pharmaceutical composition of the invention is effective within between 1 and 7 days after starting therapy. Accordingly, the compounds and compositions of the invention are formulated to provide a therapeutically effective amount of the drug after administration of a single dose. Suitably, the compounds and compositions are formulated to provide the therapeutically effective amount about 2- about 12 hours after administration of the single dose, such as about 2- about 10 hours after administration of the single dose, for example about 2- about 8 hours after administration of the single dose, or about 2- about 6 hours after administration of the single dose.

There is an accumulation effect observed with administration (such as daily dosing, or dosing every 2 days) of the compound or the pharmaceutically acceptable salt, hydrate, solvate, optical isomer, or polymorph thereof, or the pharmaceutical composition of the invention, so that Cmax increases after each dose up to around Day 10-15 after starting dosing, which is when steady state concentration is reached. Thus, suitably the use and/or methods of the invention provide an in vivo profile comprising a Cmax of between 50 and 300 μg/L of the compound at steady state concentration, preferably between 50 and 250 μg/L, more preferably between 50 and 200 μg/L, such as between 50 and 175 μg/L. Alternatively, the Cmax is between 70 and 250 μg/L, preferably 80 and 225 μg/L, such as 90 and 200 μg/L. Suitably said Cmax occurs between 10 and 15 days after the first administration of the compound or the pharmaceutically acceptable salt, hydrate, solvate, optical isomer, or polymorph thereof, preferably between 10 and 14 days, more preferably between 11 and 13 days, such as about 12 days. When steady state concentration is reached, the Cmax may occur between 2 and 8 hours after the dose, such as between 2 and 7 hours, such as between 3 and 6 hours. Suitably the in vivo profile comprising the Cmax levels derive from a brain or plasma in vivo profile, preferably brain.

Suitably the compound or the pharmaceutically acceptable salt, hydrate, solvate, optical isomer, or polymorph thereof, or the pharmaceutical composition of the invention is for use in a method of treating OCD, wherein the method provides improvement in at least one (preferably at least two, or at least three) symptom of OCD selected from the group consisting of obsessive thoughts, compulsive behaviours, anxiety and distress. Such symptoms are well-known in the field, for example, obsessive thoughts can be described as persistent, unpleasant thoughts that dominate a person's thinking to the extent it interrupts other thoughts, typically causing distress and/or has an impact on the quality of life. Compulsive behaviours typically start as a way of trying to reduce or prevent anxiety caused by the obsessive thought. For example, a person who fears contamination with germs may wash their hands repeatedly or someone with a fear of harming their family may have the urge to repeat an action multiple times to “neutralise” the thought. Anxiety is known to mean a feeling of worry, nervousness. Distress is known to mean extreme anxiety, sorrow, or pain.

Suitably the use and/or methods provide improvement in the patient, such as of the above-mentioned symptoms. Suitably, such improvements occur after at least 2 hours, such as after at least 4 hours after administration, at least 6 hours after administration, such as at least 24 hours after administration, after at least 2 days of daily administration, after at least 3 days of daily administration. As discussed above and demonstrated in the Examples whereby the rats undertook the behavioural tests 2 hours after dosing, the inventors have surprisingly found that the compounds of the invention have a fast onset of action.

The Yale-Brown Obsessive Compulsive Score (YBOCS) is the gold standard assessment scale for OCD clinical studies. The YBOCS is designed to rate symptom severity of a patient. The YBOCS provides five rating dimensions for obsessions and compulsions: time spent or occupied; interference with functioning or relationships; degree of distress; resistance; and control (i.e., success in resistance). The 10 YBOCS items are each scored on a four-point scale from 0=“no symptoms” to 4=“extreme symptoms.” The sum of the first five items is a severity index for obsessions, and the sum of the last five an index for compulsions. A translation of total score into an approximate index of overall severity is: 0-7 subclinical, 8-15 mild, 16-23 moderate, 24-31 severe, and 32-40 extreme.

The patient's OCD may be characterised by a score according to the Yale-Brown Obsessive Compulsive scale of at least 8, at least 14, at least 16, at least 24, at least 26, at least 32 or at least 35, optionally at least 16. The patient's OCD may be characterised by a Yale-Brown Obsessive Compulsive scale score of 14-40, 16-40, 24-40, 26-40, 32-40 or 35-40, preferably 16-40. Alternatively, the patient's OCD may be characterised by a Yale-Brown Obsessive Compulsive scale score of 0-13, 14-25, 26-34 or 35-40.

Suitably administration of the compound or the pharmaceutically acceptable salt, hydrate, solvate, optical isomer, or polymorph, or pharmaceutical compositions herein, results in the patient's YBOCS lowering. Suitably the lowering of the patient's YBOCS is by at least about 30%. Suitably the patient's YBOCS lowers after daily administration for at least 3 days, at least 1 week, preferably at least 10 days, more preferably at least 2 weeks, at least 17 days, at least 3 weeks, at least 24 days, at least 4 weeks, at least 5 weeks, such as at least 6 weeks.

Alternatively, or additionally, the patient's OCD may be characterised a score according to the Obsessive-Compulsive Inventory (OCI) or Obsessive-Compulsive Inventory—Revised (OCI-R) of at least 21, at least 30, at least 40, at least 50 or at least 60, preferably at least 21. The patient's OCD may be characterised a score according to the Obsessive-Compulsive Inventory (OCI) or Obsessive-Compulsive Inventory—Revised (OCI-R) of 21-72, 30-72, 40-72, 50-72 or 60-72, preferably 21-72.

Alternatively, or additionally, the patient's OCD may be characterised a score according to the Clinical Global Impression—Improvement scale (CGI-I), which is a seven-point scale that requires the clinician to assess how much the patient's illness has improved or worsened relative to a baseline state at the beginning of the intervention. The seven-point scale is: 1=very much improved, 2=much improved, 3=minimally improved, 4=no change, 5=minimally worse, 6=much worse, and 7=very much worse. Suitably administration of the compound or the pharmaceutically acceptable salt, hydrate, solvate, optical isomer, or polymorph, or pharmaceutical compositions herein, results in the patient scoring 1-3 on the CGI-I scale, preferably 1-2. Suitably the patient achieves a score of 1-3 on the CGI-I scale after daily administration for at least 3 days, at least 1 week, preferably at least 10 days, more preferably at least 2 weeks, at least 17 days, at least 3 weeks, at least 24 days, at least 4 weeks, at least 5 weeks, such as at least 6 weeks.

Alternatively, or additionally, the patient's OCD may be characterised a score according to the Sheehan Disability Scale (SDS), which measures impairment in functioning. The scale generates 4 scores: a work disability score (0-10), a social life disability score (0-10), a family life disability score (0-10) and a total score. To get a total score add up the 3 individual scores (work: social life: family life). The maximum possible score is 30 (0 unimpaired, 30 highly impaired). Scores of ≥5 on any of the 3 scales; high scores are associated with significant functional impairment. Suitably administration of the compound or the pharmaceutically acceptable salt, hydrate, solvate, optical isomer, or polymorph, or pharmaceutical compositions herein, results in the patient's SDS score lowering, preferably 1-2. Suitably the patient's SDS score lowers after daily administration for at least 3 days, at least 1 week, preferably at least 10 days, more preferably at least 2 weeks, at least 17 days, at least 3 weeks, at least 24 days, at least 4 weeks, at least 5 weeks, such as at least 6 weeks.

The patient may have compulsions. The patient may have checking compulsions.

The pharmaceutical composition may comprise a pharmaceutically acceptable excipient. The pharmaceutically acceptable excipient may be selected from the group consisting of a solvent, a co-solvent, a buffer, a stabiliser, an antioxidant, a preservative, a chelating agent, an emulsifier, a flavouring, a lubricant, a suspending agent, a tonicity-adjusting agent, a surfactant, a solubilising agent, a suspending aid, a dispersion agent, a humectant, a thickener, a colouring agent, a wetting agent, an anti-foaming agent, a viscosity modifier, a sweetener, and any combination thereof. The pharmaceutical formulation may comprise the compound or the pharmaceutically acceptable salt, hydrate, solvate, optical isomer, or polymorph thereof and phosphate buffered saline.

The pharmaceutical composition may comprise a second active agent. The second active agent may comprise an atypical neuroleptic, a neuroleptic, an anticonvulsant or an antidepressant. The atypical neuroleptic may comprise amisulpride, aripiprazole, asenapine, blonanserin, clozapine, lurasidone, melperone, olanzapine, paliperidone, perospirone, risperidone, sertindole or sulpiride. The neuroleptic may comprise chlorpromazine, chlorprothixene, levomepromazine, mesoridazine, periciazine, promazine, loxapine, molindone, perphenazine, thiothixene, droperidol, flupentixol, fluphenazine, pimozide, prochlorperazine, thioproperazine or zuclopenthixol. The anticonvulsant may comprise paraldehyde, phenobarbital, clobazam, clonazepam, clorazepate, diazepam, midazolam, a valproate, vigabatrin, progabide, tiagabine, ethadione, brivaracetam or zonisamine. The antidepressant may comprise fluoxetine, paroxetine, sertraline, citalopram, escitalopram, duloxetine, venlafaxine, desvenlafaxine, trazodone, vortioxetine, bupropion, imipramine, nortriptyline, amitriptyline, tranylcypromine or phenelzine.

In a third aspect the invention provides a kit comprising the aforementioned compound or a pharmaceutically acceptable salt, hydrate, solvate, optical isomer, or polymorph thereof. The kit may comprise instructions (for providing information to the patient, such as safety information or information on how to correctly administer the compound or pharmaceutical composition) or the aforementioned second active agent. The second active agent may be for simultaneous, separate or sequential use with the aforementioned compound or a pharmaceutically acceptable salt, hydrate, solvate, optical isomer, or polymorph thereof.

In a fourth aspect the invention provides a method of treating obsessive compulsive disorder in a patient, comprising administering to the patient a therapeutically effective amount of the aforementioned compound or a pharmaceutically acceptable salt, hydrate, solvate, optical isomer, or polymorph thereof, or the aforementioned pharmaceutical composition.

In a fifth aspect, the invention provides a use of the aforementioned compound or a pharmaceutically acceptable salt, hydrate, solvate, optical isomer, or polymorph thereof in the preparation of a medicament for the treatment of obsessive compulsive disorder.

In a specific embodiment of the invention, there is provided a pharmaceutical composition, comprising 7-methyl-5-(3-piperazin-1-ylmethyl-[1,2,4]oxadiazol-5-yl)-2-(4-trifluoromethoxybenzyl)-2,3-dihydroisoindol-1-one, or a pharmaceutically acceptable salt thereof, and one or more pharmaceutically acceptable excipients; wherein: the composition is formulated to provide a therapeutically effective amount of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof, after administration of a single dose.

A method of treating obsessive compulsive disorder (OCD), comprising:
administering a pharmaceutical composition comprising:
a therapeutically effective amount of 7-methyl-5-(3-piperazin-1-ylmethyl-[1,2,4]oxadiazol-5-yl)-2-(4-trifluoromethoxybenzyl)-2,3-dihydroisoindol-1-one, or a pharmaceutically acceptable salt thereof, and one or more pharmaceutically acceptable excipients, to a patient in need of such treatment, wherein:
the composition provides a therapeutically effective amount of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof, after administration of a single dose.

### BRIEF DESCRIPTION OF THE FIGURES

The invention will now be described with reference to the following Figures which are intended to be non-limiting. For each dose shown in the figures, the order of the bars is pre-drug baseline, drug treatment and then post-drug baseline.

FIG. 1A shows the effect of AZD-8529 on functional checking (Observing Lever Presses) of the full cohort (sign-trackers plus goal-trackers) in a rat model of OCD.

FIG. 1B shows the effect of AZD-8529 on dysfunctional checking (Extra Observing Lever Presses) of the full cohort in a rat model of OCD (sign-trackers plus goal-trackers).

FIG. 2A shows the effect of AZD-8529 on functional checking (Observing Lever Presses) in a rat model of OCD (sign-trackers).

FIG. 2B shows the effect of AZD-8529 on dysfunctional checking (Extra Observing Lever Presses) in a rat model of OCD (sign-trackers).

FIG. 3A shows the effect of AZD-8529 on functional checking (Observing Lever Presses) in control rats (goal-trackers).

FIG. 3B shows the effect of AZD-8529 on dysfunctional checking (Extra Observing Lever Presses) in control rats (goal-trackers).

FIG. 4A shows the effect of AZD-8529 on Active Lever Presses (per minute) in the full cohort (sign-trackers plus goal-trackers) in a rat model of OCD (i.e. responding on the to-be-rewarded lever).

FIG. 4B shows the effect of AZD-8529 on Inactive Lever Presses (per minute) in the full cohort (sign-trackers plus goal-trackers) in a rat model of OCD, (i.e. responding on the lever not selected to be rewarded).

FIG. 5A shows the effect of AZD-8529 on discrimination between the to-be-rewarded and not-to-be-rewarded levers when the cue light (indicating the currently rewarded lever) is on, in the full cohort (sign-trackers plus goal-trackers) in a rat model of OCD. It is desirable that this percentage remains as high as possible after drug to avert possible sedative side-effects.

FIG. 5B shows the effect of AZD-8529 on the discrimination between the to-be-rewarded and not-to-be-rewarded levers during the absence of the cue light in the full cohort (sign-trackers plus goal-trackers) in a rat model of OCD.

FIG. 6 shows the effect of AZD-8529 on rewards earned in the full cohort (sign-trackers plus goal-trackers) in a rat model of OCD.

### DETAILED DESCRIPTION OF THE INVENTION

Throughout this specification, one or more aspects of the invention may be combined with one or more features described in the specification to define distinct embodiments of the invention.

In the discussion herein, reference is made to a number of terms, which are to be understood to have the meanings accepted in the art, unless explicitly defined below.

Throughout this specification the word “comprise” will be understood to mean the inclusion of a stated element or integer, or group of elements or integers, but not the exclusion of any other element or integer, or group of elements or integers.

The term “consisting” is to be understood to mean the inclusion of a stated element or integer, or group of elements or integers, and the exclusion of any other element or integer or group of elements or integers.

The term “consisting essentially of” is to be understood to mean the inclusion of a stated element or integer, or group of elements or integers, and that further components may be present, provided that those further components do not materially affect the essential characteristics of the formulation, composition, or compound.

The term “about” herein, when qualifying a number or value, is used to refer to values that lie within +1%, 5%, or +10% of the value specified.

The terms “treatment” and “therapy” define the therapeutic treatment of a patient, but also include within their meanings the reduction or halting of the rate of progression of a disorder or condition, or the amelioration or cure the disorder or condition. Prophylaxis of a disorder or condition as a result of treatment or therapy is also included.

As used herein, the term “patient” preferably refers to a mammal. Typically, the mammal is a human.

The term “C₁₋₃ alkyl” means a straight or branched chain or cyclic hydrocarbon having one to three carbon atoms, and includes methyl, ethyl, propyl, isopropyl, and cyclopropyl.

The term “C₁₋₃ haloalkoxyl” means a straight or branched chain alkoxy having one to three carbon atoms and at least one halo substituent and includes fluoromethoxyl, trifluoromethoxyl, fluoroethoxyl, trifluropropyloxyl, fluoroisopropyloxyl and the like.

The term “halo” includes fluoro, chloro, bromo, iodo, in both radioactive and non-radioactive forms.

The term “pharmaceutically acceptable” refers to any non-toxic composition of matter that is suitable for administration to a patient. For example, the term “pharmaceutically acceptable salt” is preferably any non-toxic organic or inorganic acidic addition salt of the aforementioned compound. Illustrative inorganic acids that form suitable salts include hydrochloric, hydrobromic, sulfuric and phosphoric acid, and acid metal salts such as sodium monohydrogen orthophosphate and potassium hydrogen sulphate. Illustrative organic acids that form suitable salts include the mono-, di- and tricarboxylic acids. Illustrative of such acids are acetic, glycolic, lactic, pyruvic, malonic, succinic, glutaric, fumaric, malic, tartaric, citric, ascorbic, maleic, hydroxy maleic, benzoic, hydroxybenzoic, phenylacetic, cinnamic, salicylic, and sulfonic acids. Preferably, the salt is a hydrochloride salt, a mesylate salt or a valine salt. Even more preferably, the salt is a mesylate salt.

The term “therapeutically effective amount” means an amount of the compound that is effective in treating the named disorder or condition.

A dose measured in “mg/kg” refers to a dose of an active agent in milligrams calculated based on the body weight of a patient to be treated. For example, a dose of 1 mg/kg would result in a 100 kg subject being administered a dose of 100 mg.

### Compounds

The compounds of the present invention are described in PCT publication number WO 2008/150233 A1, where they are referred to as “compounds of Formula (I)”. WO 2008/150233 A1 describes the synthesis of the compounds of the present invention. Briefly, the compound of the present invention may be synthesised according to the following synthetic scheme:

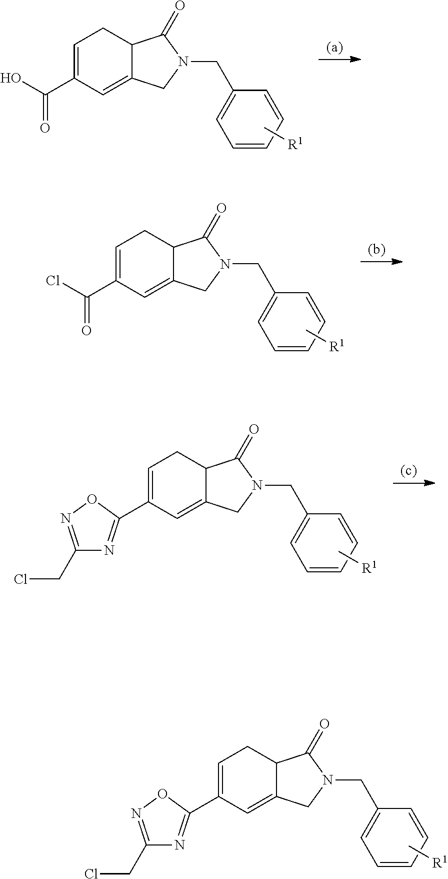

Reagents and conditions that may be used in a typical procedure: (a) SOCl₂, Δ; (b) 2-chloro-N-hydroxyacetamidine, K₂CO₃, MeCN, then DMF, Δ; (c) QH, K₂CO₃, MeCN, Δ.
(a) In a typical procedure, 100 mmol of a 7-methyl-1-oxo-2-(substituted benzyl)-2,3-dihydro-1H-isoindole-5-carboxylic acid may be dissolved in an excess of thionyl chloride and heated at reflux for 30 minutes. The reaction may be cooled to room temperature and concentrated to provide 7-methyl-1-oxo-2-(substituted benzyl)-2,3-dihydro-1H-isoindole-5-carbonyl chloride.
(b) To a solution of the 7-methyl-1-oxo-2-(substituted benzyl)-2,3-dihydro-1H-isoindole-5-carbonyl chloride (100 mmol) in MeCN (50 mL) 2-chloro-N-hydroxyacetamidine (110 mmol) and K₂CO₃ (200 mmol) may be added. The reaction mixture may be stirred overnight, then diluted with water and extracted with EtOAc. The organic phase may be washed with brine, dried with Na₂SO₄, filtered and concentrated. The residue may be dissolved in DMF (50 mL) and heated at reflux for 3.5 hours. The cooled solution may be diluted with water and extracted with EtOAc. The organic phase may be washed with brine, dried with Na₂SO₄, filtered, concentrated and purified using silica gel chromatography with 10-35% EtOAc/hexanes to provide a 2-substituted-benzyl-5-(3-chloromethyl-[1,2,4]oxadiazol-5-yl)-7-methyl-2,3-dihydro-isoindol-1-one.
(c) To a solution of the 2-substituted-benzyl-5-(3-chloromethyl-[1,2,4]oxadiazol-5-yl)-7-methyl-2,3-dihydro-isoindol-1-one (100 mmol) in MeCN K₂CO₃ (200-300 mmol) and a suitable amine (QH, 150-200 mmol) may be added. The mixture may be heated to provide a desired isoindolone that may be purified using silica gel chromatography 1-5% 2M NH₃ in MeOH/CH₂Cl₂).

The term “compound” or “compound or a pharmaceutically acceptable salt, hydrate, solvate, optical isomer or polymorph thereof” refers to a compound comprising, or having, the general structure:

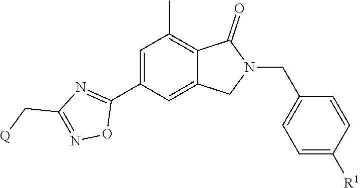

wherein
   R¹ is halo or C₁₋₃ haloalkoxyl;
Q is

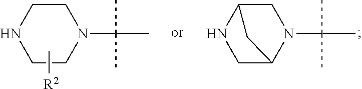

and
R² is hydrogen or C₁₋₃ alkyl.

Preferably, R¹ is chloro- or trifluoromethoxyl. More preferably R¹ is trifluoromethoxyl.

The compound may comprise, or is, 7-methyl-5-(3-piperazin-1-ylmethyl-[1,2,4]oxadiazol-5-yl)-2-(4-trifluoromethoxybenzyl)-2,3-dihydroisoindol-1-one. 7-methyl-5-(3-piperazin-1-ylmethyl-[1,2,4] oxadiazol-5-yl)-2-(4-trifluoromethoxybenzyl)-2,3-dihydroisoindol-1-one may be referred to as AZD-8529. Preferably, the compound comprises the structure:

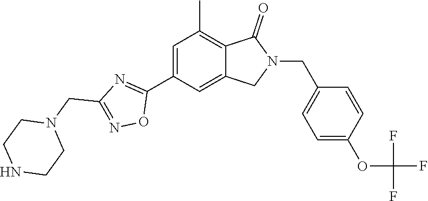 [SMILES: Cc1cc(-c2nc(CN3CCNCC3)no2)cc2c1C(=O)N(Cc1ccc(OC(F)(F)F)cc1)C2]

or a pharmaceutically acceptable salt, hydrate, solvate, optical isomer or polymorph thereof.

The salt of 7-methyl-5-(3-piperazin-1-ylmethyl-[1,2,4] oxadiazol-5-yl)-2-(4-trifluoromethoxybenzyl)-2,3-dihydroisoindol-1-one may be the mesylate salt. The mesylate salt is described in WO2011/136723, as well as processes for preparing the same. Polymorphic forms A, C and D of 7-methyl-5-(3-piperazin-1-ylmethyl-[1,2,4] oxadiazol-5-yl)-2-(4-trifluoromethoxybenzyl)-2,3-dihydroisoindol-1-one mesylate salt are also described in WO2011/136723, as well as processes for preparing the same. Accordingly the compound or polymorph or salt thereof may comprise 7-methyl-5-(3-piperazin-1-ylmethyl-[1,2,4]oxadiazol-5-yl)-2-(4-trifluoromethoxybenzyl)-2,3-dihydroisoindol-1-one mesylate salt, or polymorphic forms A, C or D thereof. Polymorphic form A is preferred.

The compound may comprise 2-(4-chloro-benzyl)-5-[3-(2,5-diaza-bicyclo[2.2.1]hept-2-ylmethyl)-[1,2,4]oxadiazol-5-yl]-7-methyl-2,3-dihydro-isoindol-1-one, or a pharmaceutically acceptable salt, hydrate, solvate, optical isomer or polymorph thereof, wherein 2-(4-chloro-benzyl)-5-[3-(2,5-diaza-bicyclo[2.2.1]hept-2-ylmethyl)-[1,2,4]oxadiazol-5-yl]-7-methyl-2,3-dihydro-isoindol-1-one has the structure:

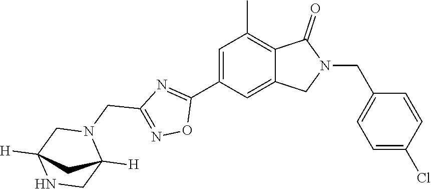 [SMILES: Cc1cc(-c2nc(CN3C[C@@H]4C[C@H]3CN4)no2)cc2c1C(=O)N(Cc1ccc(Cl)cc1)C2]

The compound may comprise 2-(4-chlorobenzyl)-7-methyl-5-[3-(3-methyl-piperazin-1-ylmethyl)-[1,2,4]oxadiazol-5-yl]-2,3-dihydro-isoindol-1-one, or a pharmaceutically acceptable salt, hydrate, solvate, optical isomer or polymorph thereof, wherein 2-(4-chlorobenzyl)-7-methyl-5-[3-(3-methyl-piperazin-1-ylmethyl)-[1,2,4]oxadiazol-5-yl]-2,3-dihydro-isoindol-1-one has the structure:

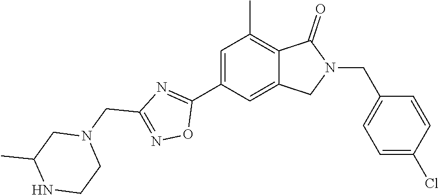 [SMILES: Cc1cc(-c2nc(CN3CCNC(C)C3)no2)cc2c1C(=O)N(Cc1ccc(Cl)cc1)C2]

The compound may comprise 2-(4-chloro-benzyl)-7-methyl-5-(3-piperazin-1-ylmethyl-[1,2,4]oxadiazol-5-yl)-2,3-dihydro-isoindol-1-one, or a pharmaceutically acceptable salt, hydrate, solvate, optical isomer or polymorph thereof, wherein 2-(4-chloro-benzyl)-7-methyl-5-(3-piperazin-1-ylmethyl-[1,2,4]oxadiazol-5-yl)-2,3-dihydro-isoindol-1-one has the structure:

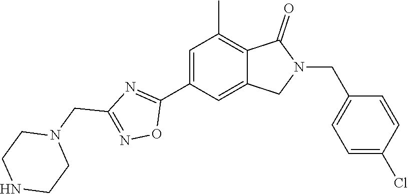 [SMILES: Cc1cc(-c2nc(CN3CCNCC3)no2)cc2c1C(=O)N(Cc1ccc(Cl)cc1)C2]

The compound may comprise 2-(4-chloro-benzyl)-7-methyl-5-[3-(2-methyl-piperazin-1-ylmethyl)-[1,2,4]oxadiazol-5-yl]-2,3-dihydro-isoindol-1-one, or a pharmaceutically acceptable salt, hydrate, solvate, optical isomer or polymorph thereof, wherein 2-(4-chloro-benzyl)-7-methyl-5-[3-(2-methyl-piperazin-1-ylmethyl)-[1,2,4]oxadiazol-5-yl]-2,3-dihydro-isoindol-1-one has the structure:

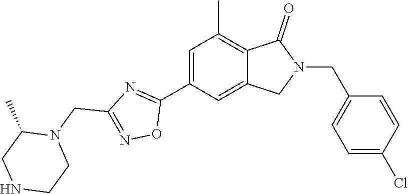 [SMILES: Cc1cc(-c2nc(CN3CCNC[C@@H]3C)no2)cc2c1C(=O)N(Cc1ccc(Cl)cc1)C2]

The compound may comprise 2-(4-chloro-benzyl)-7-methyl-5-[3-(2-methyl-piperazin-1-ylmethyl)-[1,2,4]oxadiazol-5-yl]-2,3-dihydro-isoindol-1-one, or a pharmaceutically acceptable salt, hydrate, solvate, optical isomer or polymorph thereof, wherein 2-(4-chloro-benzyl)-7-methyl-5-[3-(2-methyl-piperazin-1-ylmethyl)-[1,2,4]oxadiazol-5-yl]-2,3-dihydro-isoindol-1-one has the structure:

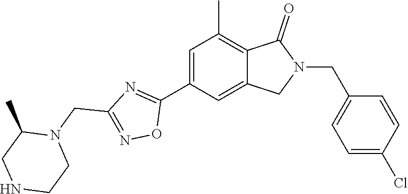 [SMILES: Cc1cc(-c2nc(CN3CCNC[C@H]3C)no2)cc2c1C(=O)N(Cc1ccc(Cl)cc1)C2]

The compounds described herein have been shown to be positive allosteric modulators of the mGluR2 receptor using a [³⁵S]-GTPγS binding assay as described in PCT international publication number WO 2008/150233 A1. The mGluR2 receptor is expressed exclusively in the synapse of the brain. Activation of this receptor results in the inhibition of the release of glutamate by the presynaptic neuron. Thus, activation of this receptor inhibits glutamate release into the synapse. The EC₅₀ values for mGluR2, taken from WO 2008/150233 A1, are as follows.

| | GTPgS EC⁵⁰ |
|---|---|
| Compound | μM |
| 7-methyl-5-(3-piperazin-1-ylmethyl-[1,2,4] oxadiazol-5-yl)- | 0.231 |
| 2-(4-trifluoromethoxybenzyl)-2,3-dihydroisoindol-1-one | |
| 2-(4-chloro-benzyl)-5-[3-(2,5-diaza-bicyclo[2.2.1]hept-2- | 0.206 |
| ylmethyl)-[1,2,4]oxadiazol-5-yl]-7-methyl-2,3-dihydro- | |
| isoindol-1-1one | |
| 2-(4-chlorobenzyl)-7-methyl-5-[3-(3-methyl-piperazin-1- | 0.154 |
| ylmethyl)-[1,2,4]oxadiazol-5-yl]-2,3-dihydro-isoindol-1-one | |
| 2-(4-chloro-benzyl)-7-mehtyl-5-(3-piperazin-1-ylmethyl- | 0.378 |
| [1,2,4]oxadiazol-5-yl)-2,3-dihydro-isoindol-1-one | |
| 2-(4-chloro-benzyl)-7-methyl-5-[3-(2-methyl-piperazin-1- | 0.352 |
| ylmethyl)-[1,2,4]oxadiazol-5-yl]-2,3-dihydro-isoindol-1-one | |
| 2-(4-chloro-benzyl)-7-methyl-5-[3-(2-methyl-piperazin-1- | 0.317 |
| ylmethyl)-[1,2,4]oxadiazol-5-yl]-2,3-dihydro-isoindol-1-one | |

### Obsessive Compulsive Disorder

In the Diagnostic and Statistical Manual of Mental Disorders, 5^{th} edition (DSM-V), OCD is recognised as a disorder distinct from anxiety. OCD is grouped with several other disorders with common features, which group is defined as “Obsessive-Compulsive and Related Disorders”. The group includes Body Dysmorphic Disorder, Hoarding Disorder and others in addition to OCD. The DSM-V provides the following diagnostic criteria for OCD:
A. Presence of obsessions, compulsions, or both.
   Obsessions are defined by (1) and (2):
      (1) Recurrent and persistent thoughts, urges, or images that are experienced, at some time during the disturbance, as intrusive and unwanted, and that in most individuals cause marked anxiety or distress.
      (2) The individual attempts to ignore or suppress such thoughts, urges, or images, or to neutralise them with some other thought or action.
   Compulsions are defined by (1) and (2):
      (1) Repetitive behaviours (e.g. hand washing, ordering, checking) or mental acts (e.g. praying, counting, repeating words silently) that the individual feels driven to perform in response to an obsession or according to rules that must be applied rigidly.
      (2) The behaviours or mental acts are aimed at preventing or reducing anxiety or distress, or preventing some dreaded event or situation; however, these behaviours or mental acts are not connected in a realistic way with what they are designed to neutralise or prevent, or are clearly excessive.
B. The obsessions or compulsions are time-consuming or cause clinically significant distress or impairment in social, occupational, or other important areas of functioning.
C. The obsessive-compulsive symptoms are not attributable to the physiological effects of a substance (e.g. a drug of abuse, a medication) or another medical condition.
D. The disturbance is not better explained by the symptoms of another mental disorder (e.g. generalised anxiety disorder).

OCD is typically diagnosed by a healthcare provider following a discussion with a patient about their symptoms. An improvement in OCD may result in a reduction in the number or time spent on obsessions and/or compulsions. The patient may generally feel less anxious as the OCD symptoms are reduced.

The severity of OCD may be measured by the Yale-Brown Obsessive Compulsive Scale. This scale is a clinician-rated, 10-item scale, each item rated from 0 (no symptoms) to 4 (extreme symptoms) (total range, 0 to 40), with separate subtotals for severity of obsessions and compulsions. The Yale-Brown Obsessive Compulsive Scale is described in Goodman et al., Arch. Gen. Psychiatry 1989; 46: 1006-1011 and Goodman et al., Arch. Gen. Psychiatry 1989; 46: 1012-1016. The patient's OCD may be characterised by a score of at least about 8, at least about 12, at least about 16, at least about 20, at least about 24, at least about 28 or at least about 32, preferably at least 16, according to the Yale Brown Obsessive Compulsive Scale. Following treatment with the aforementioned compound or a pharmaceutically acceptable salt, hydrate, solvate, optical isomer or polymorph thereof, a patient may exhibit a reduction in their Yale Brown Obsessive Compulsive score.

Alternatively, or additionally, the severity of OCD may be measured according to the Obsessive Compulsive Inventory (or its updated form: Obsessive Compulsive Inventory—Revised). The Obsessive-Compulsive Inventory consists of 42 items composing 7 subscales: Washing, Checking, Doubting, Ordering, Obsessing (i.e., having obsessional thoughts). Hoarding, and Mental Neutralizing. Each item is rated on a 5-point (0-4) Likert scale of symptom frequency and associated distress. The Obsessive-Compulsive Inventory is described in Foa et al., Psychological Assessment, 1998, 10, 3, 206-214. The patient's OCD may be characterised by a score according to the Obsessive Compulsive Inventory of at least 21, at least 30, at least 35, at least 40, at least 45, at least 50, at least 55, at least 60, at least 65 or at least 70, preferably at least 40; or a score of 21-72, 30-72, 40-72, 50-72 or 60-72, preferably 40-72. Following treatment with the aforementioned compound or a pharmaceutically acceptable salt, hydrate, solvate, optical isomer or polymorph thereof, the patient may have a reduction in their Obsessive Compulsive Inventory score.

OCD is heterogenous. Those with OCD may have a variety of obsessions and compulsions. The obsessions may be fear of harming themselves or others through their own actions, the fear of harming themselves or others through a mistake such as leaving a light on, the fear of contamination or disease, or the need for symmetry and orderliness. The compulsions may include cleaning and hand-washing, checking, counting, ordering and arranging, hoarding, asking for reassurance, or repeating words in their head.

Optionally, the patient to be treated with the aforementioned compound or a pharmaceutically acceptable salt, hydrate, solvate, optical isomer or polymorph thereof exhibits checking compulsions. Typically, checking compulsions arise when a person experiences intrusive thoughts, fears of or concerns about, for example, items not being where they are supposed to be, lights or appliances being left on, and doors not being locked. As a result of these obsessions, patients with OCD feel the compulsion to “check” and make sure the items they are concerned with have been put in the right place, the lights or appliances are turned off, and the doors are locked. The presence of checking compulsions can be determined through conversations with the patient by a healthcare provider.

OCD may be studied using animal models and such models may be used to evaluate compounds of the present invention. One model may be a rat model of compulsive-like checking behaviour described by Eagle et al., Behav. Brain Res, 1 May 2014; 264(100): 207-29. Briefly, rats are trained to work for palatable food which is unpredictably available for responding on one of two levers in an operant chamber. The rats can gain information about the location of the effective lever by making ‘observing responses’ on a third lever at the rear of the chamber. Predictably, responding on this lever is increased under conditions of uncertainty, as this adaptive checking increases the chance of food reward. However, some rats respond excessively on the observing lever and this can be disadvantageous or ‘non-functional’. This excessive checking behaviour is boosted by treatment with dopamine D2 receptor drugs such as quinpirole and also if there is a prior tendency in rats to over-respond to conditioning signals for food (‘sign-tracking’), associated with elevated dopamine activity in the nucleus accumbens. Rats who respond to the food itself are called goal-trackers. Hence, the sign-tracking rats can be regarded as providing a model of checking in OCD, and the goal-trackers can serve as controls. Moreover, in a human version of this test (i.e. involving essentially the same task for humans as for rodents), it has been shown that OCD patients check more than healthy control volunteers (Morein-Zamir et al., Q. J. Exp. Psychol. (Hove), 2018 October; 71(10): 2052-2069). In particular, this study showed that a group of OCD patients with a mixture of symptoms showed a significant increase in checking overall. Thus, as well as being a prominent compulsion observed in OCD, it is also increased even in patients for whom checking is not their primary compulsion/obsession. Hence, checking is an important behavioural biomarker of the compulsive tendency and this animal model is representative of OCD in humans.

### Dose

The aforementioned compound or the pharmaceutically acceptable salt, hydrate, solvate, optical isomer or polymorph thereof may be administered at a dose of at least 0.1 mg/kg, at least 0.3 mg/kg, at least 0.5 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg, at least 11 mg/kg, at least 12 mg/kg, at least 13 mg/kg, at least 14 mg/kg, at least 15 mg/kg, at least 16 mg/kg, at least 17 mg/kg, at least 18 mg/kg, at least 19 mg/kg, at least 20 mg/kg, at least 25 mg/kg, at least 30 mg/kg, at least 35 mg/kg, at least 40 mg/kg, at least 45 mg/kg, or at least 50 mg/kg. The aforementioned compound or the pharmaceutically acceptable salt, hydrate, solvate, optical isomer or polymorph thereof may be administered at a dose of at most 1 mg/kg, at most 2 mg/kg, at most 0.5 mg/kg, at most 4 mg/kg, at most 5 mg/kg, at most 6 mg/kg, at most 7 mg/kg, at most 8 mg/kg, at most 9 mg/kg, at most 10 mg/kg, at most 11 mg/kg at most 12 mg/kg, at most 13 mg/kg, at most 14 mg/kg, at most 15 mg/kg, at most 16 mg/kg, at most 17 mg/kg, at most 18 mg/kg, at most 19 mg/kg, at most 20 mg/kg, at most 25 mg/kg, at most 30 mg/kg, at most 35 mg/kg, at most 40 mg/kg, at most 45 mg/kg, or at most 50 mg/kg. Preferably, the compound comprises 7-methyl-5-(3-piperazin-1-ylmethyl-[1,2,4] oxadiazol-5-yl)-2-(4-trifluoromethoxybenzyl)-2,3-dihydroisoindol-1-one.

The aforementioned compound or the pharmaceutically acceptable salt, hydrate, solvate, optical isomer or polymorph thereof may be administered at a dose of 0.01 mg/kg to 100 mg/kg, 0.1 mg/kg to 50 mg/kg, 0.5 mg/kg to 45 mg/kg, 0.5 mg/kg to 40 mg/kg, 1 mg/kg to 30 mg/kg or 2 mg/kg to 20 mg/kg, 0.5 mg/kg to 10 mg/kg, or preferably 3 mg/kg to 10 mg/kg. The aforementioned compound or the pharmaceutically acceptable salt, hydrate, solvate, optical isomer or polymorph thereof may be administered at a dose of 0.01 mg/kg to 100 mg/kg, 0.05 mg/kg to 50 mg/kg, 0.1 mg/kg to 30 mg/kg, 0.5 mg/kg to 10 mg/kg, or preferably 1 mg/kg to 3 mg/kg. Preferably, the compound comprises 7-methyl-5-(3-piperazin-1-ylmethyl-[1,2,4] oxadiazol-5-yl)-2-(4-trifluoromethoxybenzyl)-2,3-dihydroisoindol-1-one. Preferably, the compound comprises 7-methyl-5-(3-piperazin-1-ylmethyl-[1,2,4] oxadiazol-5-yl)-2-(4-trifluoromethoxybenzyl)-2,3-dihydroisoindol-1-one and is administered at a dose of 0.5 mg/kg or 10 mg/kg.

Whereas sometimes the dose to be administered to an animal varies linearly according to the weight of the animal, scaling of a dose between species may alternatively be achieved using the principles of allometric scaling. The basis of allometric scaling lies in the relationship between metabolic rate (defined as the rate of biological life processes and metabolism) and the body size of the animal (Nair and Jacob, 2016, doi: 10.4103/0976-0105.177703). According to allometric scaling, the human dose in mg/kg is roughly one-sixth of the dose in mg/kg administered to a rat.

The dose may be administered over a period of time. The period of time may be from 1 day to 1 week, 1 day to 1 month, 1 month to 12 months, for example from 2 months to 11 months, from 3 months to 10 months, from 4 months to 9 months, from 5 months to 8 months, from 6 months to 7 months. The dose may be administered for at least 1 month, at least 2 months, at least 3 months, at least 4 months, at least 5 months, at least 6 months, at least 7 months, at least 8 months, at least 9 months, at least 10 months, at least 11 months or at least 12 months. The dose may be administered for 1 year, 2 years, 3 years, 4 years or at least 5 years. The dose may be administered for a suitable amount of time such that the symptoms of OCD decrease. The dose may be administered for an indefinite period of time (e.g. for the duration of the patient's life). Preferably, the aforementioned compound or the pharmaceutically acceptable salt, hydrate, solvate, optical isomer or polymorph thereof is administered for at least 3 months and even more preferably for at least one year. Even more preferably, the aforementioned compound or the pharmaceutically acceptable salt, hydrate, solvate, optical isomer or polymorph thereof is administered for an indefinite period of time (e.g. for the duration of the patient's life). Preferably, the compound comprises 7-methyl-5-(3-piperazin-1-ylmethyl-[1,2,4]oxadiazol-5-yl)-2-(4-trifluoromethoxybenzyl)-2,3-dihydroisoindol-1-one.

The dose may be administered once a day, twice a day, three times a day or four times a day. The dose may be administered once a week, twice a week, three times a week, four times a week, five times a week, six times a week, or 7 times a week. Preferably, a dose of 0.5 mg/kg to 10 mg/kg is administered once a day, twice a day, three times a day or four times a day. Preferably, the aforementioned compound is 7-methyl-5-(3-piperazin-1-ylmethyl-[1,2,4]oxadiazol-5-yl)-2-(4-trifluoromethoxybenzyl)-2,3-dihydroisoindol-1-one. Preferably, the aforementioned compound is 7-methyl-5-(3-piperazin-1-ylmethyl-[1,2,4]oxadiazol-5-yl)-2-(4-trifluoromethoxybenzyl)-2,3-dihydroisoindol-1-one and is administered once a day. More preferably, the aforementioned compound is 7-methyl-5-(3-piperazin-1-ylmethyl-[1,2,4]oxadiazol-5-yl)-2-(4-trifluoromethoxybenzyl)-2,3-dihydroisoindol-1-one and is administered once a day at a dose of 0.5 mg/kg-10 mg/kg.

The aforementioned compound or the pharmaceutically acceptable salt, hydrate, solvate, optical isomer or polymorph thereof may be administered parenterally, subcutaneously, subcutaneously, intravenously, intramuscularly, intrathecally, intradermally, intraarterially, intraarticularly, intraperitoneally, via cutaneous administration, via transcutaneous administration, via intra-osseus administration or by inhalation. Preferably, the aforementioned compound or the pharmaceutically acceptable salt, hydrate, solvate, optical isomer or polymorph thereof is administered orally. Preferably, the aforementioned compound is 7-methyl-5-(3-piperazin-1-ylmethyl-[1,2,4]oxadiazol-5-yl)-2-(4-trifluoromethoxybenzyl)-2,3-dihydroisoindol-1-one. A dose of 0.5 mg/kg to 10 mg/kg of 7-methyl-5-(3-piperazin-1-ylmethyl-[1,2,4] oxadiazol-5-yl)-2-(4-trifluoromethoxybenzyl)-2,3-dihydroisoindol-1-one may be administered orally. A dose of 0.5 mg/kg to 10 mg/kg of 7-methyl-5-(3-piperazin-1-ylmethyl-[1,2,4]oxadiazol-5-yl)-2-(4-trifluoromethoxybenzyl)-2,3-dihydroisoindol-1-one may be administered orally and once a day, twice a day, three times a day or four times a day. A dose of 0.5 mg/kg to 10 mg/kg of 7-methyl-5-(3-piperazin-1-ylmethyl-[1,2,4]oxadiazol-5-yl)-2-(4-trifluoromethoxybenzyl)-2,3-dihydroisoindol-1-one may be administered orally and once a day, twice a day, three times a day or four times a day, for at least 3 months.

### Pharmaceutical Composition

A pharmaceutical composition is any composition that is suitable for administration to a patient. The patient is typically a human. The pharmaceutical composition may comprise the aforementioned compound or the pharmaceutically acceptable salt, hydrate, solvate, optical isomer or polymorph thereof and preferably a pharmaceutically acceptable excipient. Preferably, the aforementioned compound is 7-methyl-5-(3-piperazin-1-ylmethyl-[1,2,4]oxadiazol-5-yl)-2-(4-trifluoromethoxybenzyl)-2,3-dihydroisoindol-1-one. The pharmaceutically acceptable excipient may comprise a solvent, a co-solvent, a buffer, a stabiliser, an antioxidant, a preservative, a chelating agent, an emulsifier, a flavouring, a lubricant, a suspending agent, a tonicity adjusting agent, a surfactant, a solubilising agent, a suspending aid, a dispersion agent, a humectant, a thickener, a colouring agent, a wetting agent, an anti-foaming agent, a viscosity modifier, a sweetener or any combination thereof. The pharmaceutically acceptable excipient may comprise glucose. Preferably, the pharmaceutically acceptable excipient comprises sodium chloride, for example a 0.9% (w/v) physiological saline solution. The pharmaceutically acceptable excipient may comprise phosphate buffered saline.

The pharmaceutical composition may comprise the aforementioned compound or the pharmaceutically acceptable salt, hydrate, solvate, optical isomer or polymorph thereof in an amount of 7 mg, 10 mg, 15 mg, 20 mg, 21 mg, 25 mg, 30 mg, 40 mg, 50 mg, 60 mg, 80 mg, 90 mg, 95 mg, 97 mg, 100 mg, 150 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, or 800 mg. The pharmaceutical composition may comprise the aforementioned compound or the pharmaceutically acceptable salt, hydrate, solvate, optical isomer or polymorph thereof in an amount of 0.7 mg to 7000 mg, 3.5 mg to 3500 mg, 7 mg to 2100 mg, 21 mg to 700 mg, 30 mg to 210 mg, preferably 35 mg to 210 mg. Preferably, the aforementioned compound is 7-methyl-5-(3-piperazin-1-ylmethyl-[1,2,4] oxadiazol-5-yl)-2-(4-trifluoromethoxybenzyl)-2,3-dihydroisoindol-1-one. Preferably, the dose is about 35 mg or about 700 mg, or 35 mg to 210 mg. Such quantities are useful for a unit dosage form.

The pharmaceutical composition may be either in liquid or solid form. A solid form pharmaceutical composition may comprise a powder, a tablet, a dispersible granule, a capsule, a cachet or a suppository. The solid excipient may comprise a diluent, a flavouring agent, solubilising agent, lubricant, a suspending agent, a binder or a tablet disintegrating agent. The solid excipient may comprise magnesium carbonate, magnesium stearate, talc, lactose, sugar, pectin, dextrin, starch, methyl cellulose, sodium carboxymethyl cellulose, wax or the like. The solid form pharmaceutical composition may be suitable for oral administration in the form of a tablet, powder or capsule. A liquid form pharmaceutical composition may comprise a solution, a suspension or an emulsion. For example, the aforementioned compound or the pharmaceutically acceptable salt, hydrate, solvate, optical isomer or polymorph thereof may be prepared in sterile water or a water/propylene glycol solution. An aqueous solution may be prepared by dissolving the aforementioned compound or the pharmaceutically acceptable salt, hydrate, solvate, optical isomer or polymorph thereof in water and adding an excipient comprising a diluent, a flavouring agent, a solubilising agent, a lubricant or a suspending agent. The aqueous solution may be suitable for oral administration or for injection. The aqueous solution may comprise phosphate buffered saline. The aqueous solution may comprise 0.9% (w/v) physiological saline solution.

The pharmaceutical composition may comprise an additional active agent. The additional active agent may comprise an atypical neuroleptic, a neuroleptic, an anticonvulsant or an antidepressant.

The atypical neuroleptic may comprise amisulpride, aripiprazole, asenapine, blonanserin, clozapine, lurasidone, melperone, olanzapine, paliperidone, perospirone, risperidone, sertindole or sulpiride.

The neuroleptic may comprise chlorpromazine, chlorprothixene, levomepromazine, mesoridazine, periciazine, promazine, loxapine, molindone, perphenazine, thiothixene, droperidol, flupentixol, fluphenazine, pimozide, prochlorperazine, thioproperazine or zuclopenthixol.

The anticonvulsant may comprise paraldehyde, phenobarbital, clobazam, clonazepam, clorazepate, diazepam, midazolam, a valproate, vigabatrin, progabide, tiagabine, ethadione, brivaracetam or zonisamine.

The antidepressant may comprise fluoxetine, paroxetine, sertraline, citalopram, escitalopram, duloxetine, venlafaxine, desvenlafaxine, trazodone, vortioxetine, bupropion, imipramine, nortriptyline, amitriptyline, tranylcypromine or phenelzine.

The invention will now be illustrated by the following Examples which are in no way meant to be limiting.

### EXAMPLES

### Example 1—AZD-8529 Reduces Checking in a Rat Model of OCD

Male Lister-hooded rats (n=48, Charles River, UK) were group-housed in fours and maintained at approximately 95% free-feeding weight. Experiments were conducted during the dark phase of a reversed 12-hour light-dark cycle (lights off at 07:00). This research was conducted on UK Home Office Project Licence PA9FBFA9F and was regulated under the Animals (Scientific Procedures) Act 1986 Amendment Regulations 2012 following ethical review by the University of Cambridge Animal Welfare and Ethical Review Body.

The 48 male Lister-Hooded rats were trained on the Observing Response Task (ORT) and classified as sign-trackers (n=8) or goal-trackers (n=24) following AutoShaping training, as described previously [1,2]. Based on the research described above, the sign-tracker rats provide the OCD model and the goal-trackers are controls. Briefly, rats were trained in twelve operant conditioning chambers (Med Associates, Vermont, USA) to discriminate active (reinforced) and inactive (non-reinforced) levers on a variable interval (VI10-20) schedule of reinforcement. Active and inactive levers switched position on a variable time (VT70s, range 20s-120s) schedule.

Animals showing an intermediate phenotype between sign-tracking and goal-tracking continued to undergo behavioural sessions but did not receive drug.

Following the establishment of stable levels of responding on the ORT, rats underwent two sessions of the ORT on consecutive days to establish pre-drug baseline levels of responding, followed by one ORT session under the influence of AZD-8529, followed by two additional days of drug-free ORT sessions to establish a post-drug baseline. AZD-8529 mesylate salt was purchased from MedChemExpress and dissolved in 0.9% (w/v) physiological saline to the required dose. The ‘drug treatment’ data refer to behaviour under the influence of the specific dose (3 mg/kg or 10 mg/kg), 2 hours after an intraperitoneal injection. The ‘0 mg/kg’ data refer to an intraperitoneal injection of the 0.9% (w/v) physiological saline vehicle.

Rats were divided into groups to receive different doses of AZD-8529 in a counterbalanced order. The original experimental plan, based on Justinova et al. (2015)[3], involved a single intraperitoneal administration of saline vehicle control (0.9% (w/v) saline) (termed “0 mg/kg” throughout the Figures), or single doses of AZD-8529 at 10 mg/kg and 30 mg/kg in the saline vehicle. However, following unexpected adverse gastrointestinal effects in rats receiving the 30 mg/kg dose, the experimental plan was modified such that the experiment continued with doses of 3 mg/kg and 10 mg/kg of AZD-8529 and control (i.e. 0.9% (w/v) saline vehicle control). “mg/kg” refers to a dose that was determined based on the body weight of the rat. For example, a rat weighing 0.5 kg receiving a dose of 3 mg/kg would receive a dose of 1.5 mg.

All statistical analyses were conducted using IBM SPSS Statistics v27. Data were analysed using repeated measures ANOVAs. Omnibus ANOVAs were first conducted to determine whether the order of drug dosing influenced the behavioural outcomes, and whether there were differences in baseline levels of responding. Planned ANOVAs assessing the drug administration sessions only were subsequently conducted.

### Example 2—AZD-8529 Reduced Functional Checking (OLPs) in a Dose-Dependent Manner

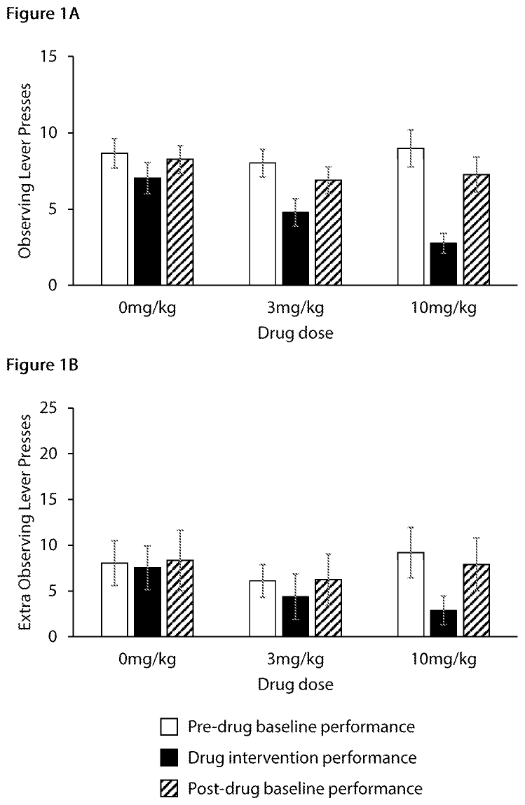

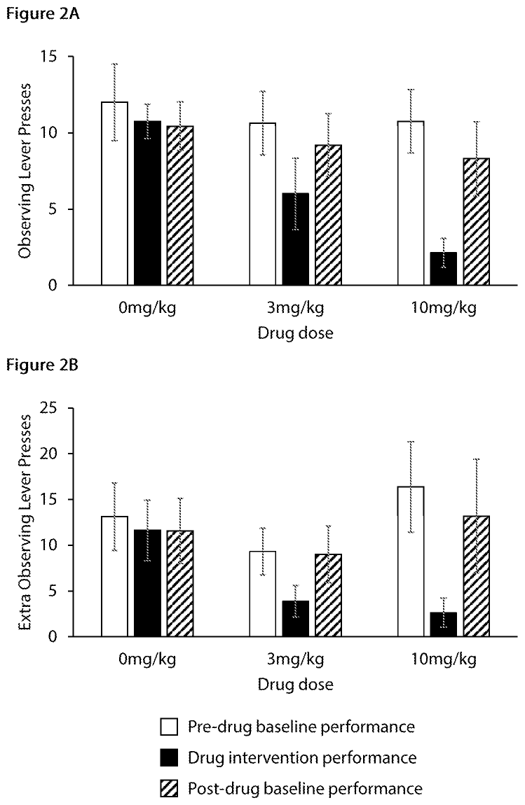

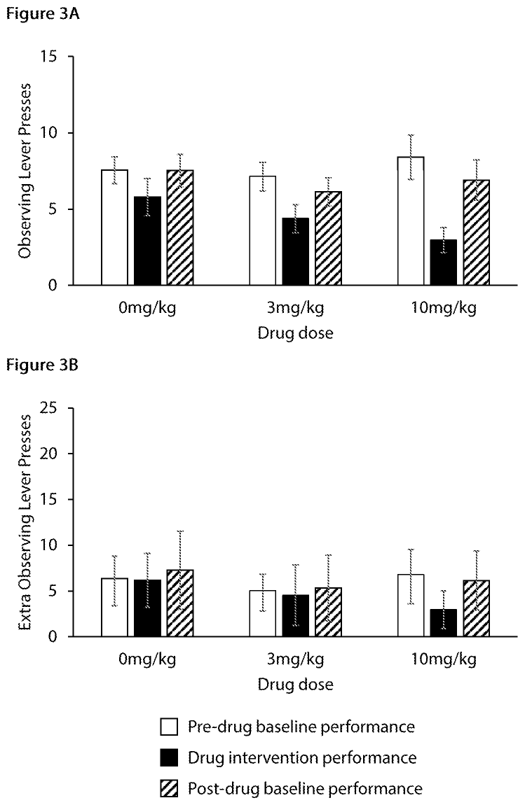

The order of drug dosing did not affect the outcome of the analysis [Order: F<1] and there were no significant interactions involving the Order factor [all F's<1] so this factor was removed from the model. FIG. 1A (OLP, i.e. a functional observing lever press) shows that functional checking was lower in the drug administration session compared to the baseline sessions [Dose×Session: F(3.7, 110)=6.73, p<0.001], with no differences between the pre- and post-drug administration baseline sessions [all p's>0.21]. A repeated measures ANOVA of the drug administration sessions alone revealed that AZD-8529 reduced functional checking in a dose-dependent manner [Dose: F(2,60)=17.3, p<0.001, η2=0.37]. FIG. 2A shows that AZD-8529 was especially effective in the OCD model (sign-trackers) [Dose×Phenotype: F(2,60)=4.45, p=0.016, η2=0.13], which showed higher levels of checking than controls (goal-trackers) in the control (“0 mg/kg”) condition [Sidak-corrected pairwise comparisons, p=0.033], but under the influence of 3 mg/kg and 10 mg/kg AZD-8529 did not differ in checking from goal-trackers [all p's>0.44]. Overall, FIGS. 2A and 3A show that AZD-8529 reduced functional checking in a dose-dependent manner in both the OCD model [p<0.001] and in controls [p=0.008].

### Example 3—AZD-8529 Reduced Dysfunctional Checking (eOLPs) in a Dose-Dependent Manner

The omnibus ANOVA revealed no overall effect of the order of drug dosing on the outcome of the analysis [Order: F<1], so this factor was removed from the model. FIG. 1B (eOLP, i.e. a dysfunctional excessive observing lever press) shows that checking was lower in the drug administration session compared to the baseline sessions [Dose×Session: F(2.9, 88)=5.97, p=0.001, η2=0.17]. The pre-drug baseline administration session prior to the 3 mg/kg dose was lower than for the sessions prior to the 0 mg/kg and 10 mg/kg doses [p=0.038] but the post-drug administration baseline sessions did not differ [all p's>0.22]. A repeated measures ANOVA of the drug administration sessions alone revealed that AZD-8529 also reduced dysfunctional checking in a dose-dependent manner [Dose: F(2,60)=11.6, p<0.001, η2=0.28]. FIG. 2B (eOLPS) shows that for dysfunctional checking, AZD-8529 was especially effective in the OCD model (sign-trackers) [Dose×Phenotype: F(2,60)=3.36, p=0.042]. Although there was no overall reduction in dysfunctional checking in controls (goal-trackers) [all p's>0.079] (FIG. 3B—eOLPS), this is likely due to their low baseline levels of dysfunctional checking. For the OCD model (sign-trackers), which showed higher levels of dysfunctional checking (as observed previously), FIG. 2B (eOLPs) shows that there was a reduction in dysfunctional checking at both the 3 mg/kg and 10 mg/kg doses of AZD-8529 compared to the 0 mg/kg control condition [all p's<0.002] but the 10 mg/kg dose did not reduce dysfunctional checking lower than the 3 mg/kg dose [p=0.94].

### Example 4—AZD-8529 Reduced Lever Pressing in the Full Cohort Only at the 10 mg/kg Dose

Measures of lever pressing provide indices of possible side-effects on the general performance of the animals (such as a sedative side-effects). Lack of impairment is indicated by relatively higher values for active lever-pressing (and lower levels for inactive lever pressing).

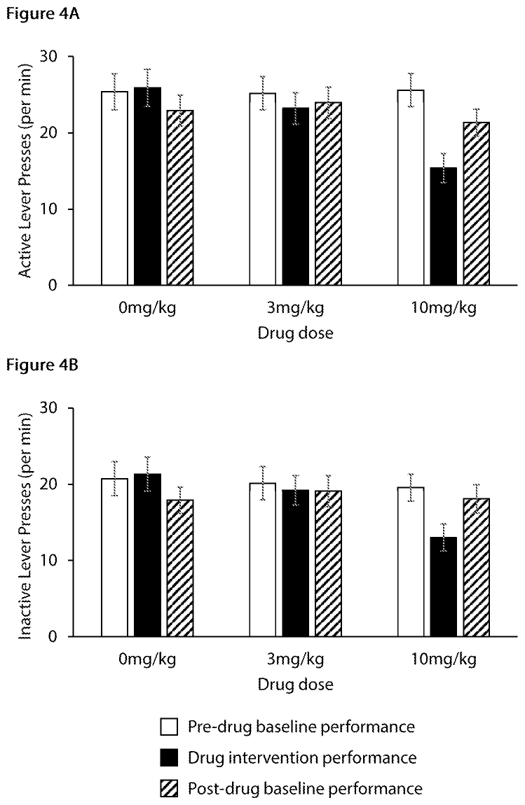

Rats discriminated between the active (ALP; “active lever pressing”) and inactive (ILP; “inactive lever pressing”) levers, with higher rates of pressing on the active lever at all doses [Lever: F(1,60)=12.3, p=0.001, η2=0.29] (FIGS. 4A and 4B). There was an overall effect of the drug dose on the rate of lever pressing [Dose: F(1.56, 43.5)=33.2, p<0.001, η2=0.53], with different effects on the OCD model (sign-trackers) and controls (goal-trackers) [Dose×Phenotype: F(1.56, 43.5)=6.14, p=0.008, η2=0.17] such that sign-trackers pressed more than goal-trackers at the 0 mg/kg and 3 mg/kg doses (all p's<0.008) but both groups showed equal rates of lever pressing at the 10 mg/kg dose (p=0.42). (Data showing differences between these sub-groups are not shown). Sidak-corrected pairwise comparisons revealed that there was no overall reduction in lever pressing at the 3 mg/kg dose for both the OCD model (sign-trackers) (p=0.18) and controls (goal-trackers) (p=0.10), but that lever pressing rates were lower at 10 mg/kg for both the OCD model (sign-trackers) (p's<0.001 compared to vehicle and the 3 mg/kg dose) and controls (goal-trackers) (p's<0.02 compared to vehicle and the 3 mg/kg dose). Thus, the 10 mg/kg acutely reduced rates of lever pressing.

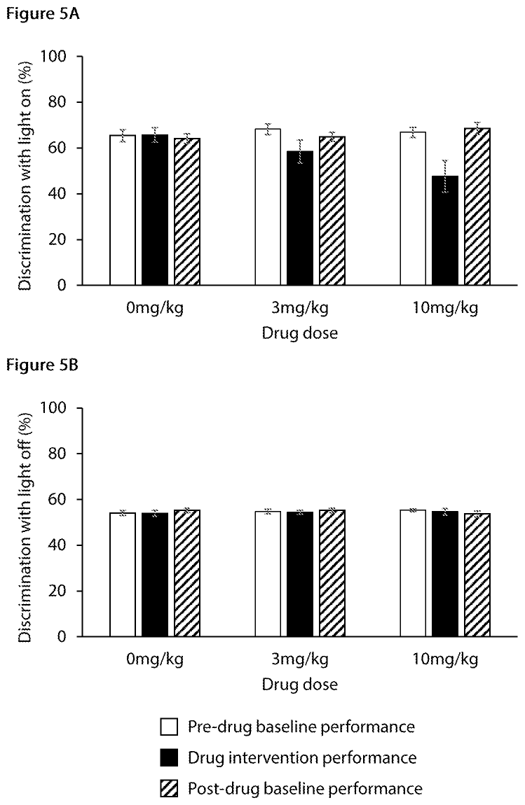

### Example 5—Lever Pressing During the Cue (‘Discrimination with Light On’) was Reduced Under AZD-8529, Likely Due to Reduced Rates of Checking (FIGS. 5A and 5B)

“Discrimination with the light on” and “Discrimination with the light off” also provide measures of possible drug side-effects on the general performance of the animals (such as a sedative side-effects). “Discrimination with the light on” should ideally be high, denoting efficiency; it is a measure of the discriminative accuracy of the rodent towards the illuminated (to be rewarded) lever. “Discrimination with the light off” is likely to be at chance (50%), reflecting task difficulty. The omnibus ANOVA revealed that rats were able to direct responding to the active lever more readily in the presence of the cue [CS: F(1,23)=13.7, p=0.001, η2=0.37]. Analysis of the drug treatment sessions only revealed a marginal effect of the drug dose [Dose: F(2, 60)=3.12, p=0.051, η2=0.09], with reduced responding during the CS [CS×Dose: F(2,60)=3.77, p=0.029, η2=0.11]. Sidak-corrected pairwise comparisons revealed that the increased responding on the active lever during vehicle treatment (p=0.007) was not seen when rats received AZD-8529 at 3 mg/kg (p=0.38) or 10 mg/kg (p=0.21), though this may be due to the reduced opportunity to respond in the presence of the cue, due to the reductions in checking behaviour at both the 3 mg/kg and 10 mg/kg doses.

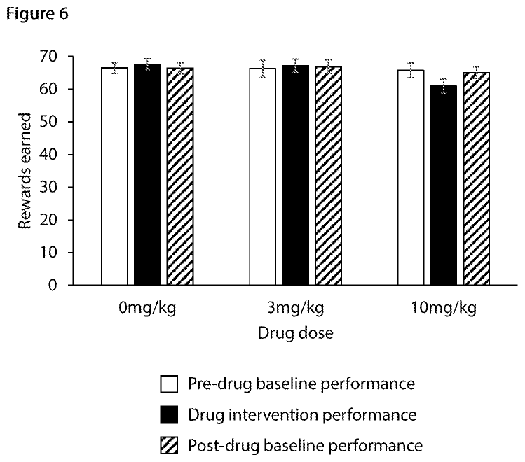

### Example 6—AZD-8529 Reduced the Numbers of Rewards Earned Only at the 10 mg/kg Dose (FIG. 6)

This is another index of non-checking performance to provide an additional index of possible side-effects.

The number of rewards earned during the session was affected by the AZD-8529 dose received [Dose: F(2,60)=20.6, p<0.001]. However, the reduction in reward number was specific to the 10 mg/kg dose, which reduced the numbers of reward earned compared to both vehicle and the 3 mg/kg dose [all p's<0.001]. By contrast, equivalent numbers of rewards were earned during vehicle treatment and treatment with the 3 mg/kg dose [p=0.91].

### Summary of Examples

Both 3 mg/kg and 10 mg/kg AZD-8529 reduced functional and dysfunctional checking behaviour, particularly in the OCD model population (of sign-trackers), which display higher baseline rates of checking. While the 10 mg/kg dose affected a number of secondary measures of task performance (rates of lever pressing and rewards earned), the 3 mg/kg dose did not appear to have the same effects on task performance. The 3 mg/kg dose acutely reduced discriminated responding on the active lever during presentation of the cue, but this reduction may be a consequence of the reduced checking (and therefore fewer opportunities to respond during the cue). Overall, the 3 mg/kg dose of AZD-8529 reduces checking without producing generalised impairments on task performance. According to direct scaling, a similar dose of 3 mg/kg dose, which has been shown to be therapeutically and selectively effective in the rat OCD model described herein, may exhibit similar therapeutic effectiveness in treating human patients with OCD. Alternatively, according to the principles of allometric scaling, a dose of about 0.5 mg/kg may exhibit similar therapeutic effectiveness in treating human patients with OCD.

### REFERENCES

1. Eagle D M, Schepisi C, Chugh S, Desai S, Han S, Huang T, et al. Dissociable dopaminergic and pavlovian influences in goal-trackers and sign-trackers on a model of compulsive checking in OCD. Psychopharmacology. 2020: doi: 10.1007/s00213-020-5636-3.
2. Vousden G H, Paulcan S, Robbins T W, Eagle D M, Milton A L. Checking responses of goal- and sign-trackers are differentially affected by threat in a rodent analogue of obsessive-compulsive disorder. Learning & Memory. 2020; 27(5):190-200.
3. Justinova Z, Panlilio L V, Secci M E, Redhi G H, Schindler C W, Cross A J, et al. The novel metabotropic glutamate receptor 2 positive allosteric modulator, AZD-8529, decreases nicotine self-administration and relapse in squirrel monkeys. Biological Psychiatry. 2015; 78:452-62.

What is claimed is:

1. A pharmaceutical composition, comprising 7-methyl-5-(3-piperazin-1-ylmethyl-[1,2,4]oxadiazol-5-yl)-2-(4-trifluoromethoxybenzyl)-2,3-dihydroisoindol-1-one, or a pharmaceutically acceptable salt thereof, and one or more pharmaceutically acceptable excipients; wherein:
the composition is formulated to provide a therapeutically effective amount of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof, after administration of a single dose.

2. The pharmaceutical composition of claim 1, wherein the composition is formulated to provide the therapeutically effective amount about 2- about 12 hours after administration of the single dose.

3. The pharmaceutical composition of claim 1, wherein the composition is formulated for administration once per day.

4. The pharmaceutical composition of claim 3, wherein the composition is formulated to provide a steady-state concentration of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof, after about 10- about 15 days of daily dosing.

5. The pharmaceutical composition of claim 4, wherein the composition is formulated to provide an in vivo profile comprising a Cmax of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof, between 50 and 300 μg/L at steady state.

6. The pharmaceutical composition of claim 1, which is formulated for oral administration.

7. The pharmaceutical composition of claim 1, which comprises 20 mg-210 mg of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof.

8. A method of treating obsessive compulsive disorder (OCD), comprising:
administering a pharmaceutical composition comprising:
a therapeutically effective amount of 7-methyl-5-(3-piperazin-1-ylmethyl-[1,2,4]oxadiazol-5-yl)-2-(4-trifluoromethoxybenzyl)-2,3-dihydroisoindol-1-one, or a pharmaceutically acceptable salt thereof, and
one or more pharmaceutically acceptable excipients,
to a patient in need of such treatment, wherein:
the composition provides a therapeutically effective amount of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof, after administration of a single dose.

9. The method of claim 8, wherein the composition treats a symptom of OCD in the patient selected from the group consisting of obsessive thoughts, compulsive behaviours, anxiety, distress, checking compulsions, and combinations thereof.

10. The method of claim 9, wherein the symptom is obsessive thoughts or checking compulsions.

11. The method of claim 8, wherein, prior to treatment, the patient has a Yale-Brown Obsessive Compulsive Score selected from the group consisting of 16-40, 24-40, and 32-40.

12. The method of claim 11, wherein the composition reduces the patient's Yale-Brown Obsessive Compulsive Score by at least about 30%.

13. The method of claim 8, wherein the composition provides the therapeutically effective amount about 2-about 12 hours after administration of the single dose.

14. The method of claim 13, wherein the therapeutically effective amount comprises 0.01 mg/kg-100 mg/kg of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof.

15. The method of claim 8, wherein the composition is administered once per day.

16. The method of claim 8, wherein the composition provides a steady-state concentration of 7-methyl-5-(3-piperazin-1-ylmethyl-[1,2,4]oxadiazol-5-yl)-2-(4-trifluoromethoxybenzyl)-2,3-dihydroisoindol-1-one, or a pharmaceutically acceptable salt thereof, in the patient after about 10- about 15 days of daily dosing.

17. The method of claim 16, wherein the composition provides an in vivo profile in the patient comprising a Cmax of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof, between 50 and 300 μg/L at steady state.

18. The method of claim 8, wherein the composition is administered orally.

19. The method of claim 8, wherein the composition comprises 20 mg-210 mg of the 7-methyl-5-(3-piperazin-1-ylmethyl-[1,2,4]oxadiazol-5-yl)-2-(4-trifluoromethoxybenzyl)-2,3-dihydroisoindol-1-one, or pharmaceutically acceptable salt thereof.
